(19) United States
(12) Patent Application Publication
(10) Pub. No.: US 2021/0035671 A1
(43) Pub. Date: Feb. 4, 2021

(54) SYSTEM AND METHOD OF DETERMINING A PRESCRIPTION FOR A PATIENT

(71) Applicant: MEDIEDU OY, Helsinki (FI)

(72) Inventor: Pauli Puirava, Helsinki (FI)

(21) Appl. No.: 16/969,065

(22) PCT Filed: Feb. 13, 2019
(86) PCT No.: PCT/FI2019/050113

§ 371 Date: Aug. 11, 2020

Related U.S. Application Data
(63) Continuation of application No. 15/899,537, filed on Feb. 20, 2018, now abandoned.

(51) Int. Cl. G16H 20/10; G06N 20/00

(52) U.S. Cl. CPC G16H 20/10; G06N 20/00

(57) ABSTRACT

A method of determining a prescription of two or more medicines for a patient, using a machine learning model executed on a server. The method includes obtaining a medical record of a new patient; generating a medicine intake matrix by obtaining medicine intake data of two or more medicines in use by the new patient; generating a symptom level matrix by obtaining levels of symptoms associated with the two or more medicines from the new patient after intake; and determining, using a transfer function, a prescription including an amount of a first medicine and an amount of a second medicine for the new patient to reduce a value of a sum of the symptom level matrix, wherein the transfer function provides an output value of prescription for the new patient based on the medicine intake matrix and the symptom level matrix.

### TECHNICAL FIELD

The present disclosure relates generally to a system and a method of determining a prescription of two or more medicines for a patient using a machine learning model; moreover, the aforesaid system employs, when in operation, machine learning techniques for determining the prescription for the patient.

### BACKGROUND

A prescription typically includes written instructions given by a health care practitioner for a patient to consume two or more medicines (e.g. drugs). Each medicine may independently have its own side effects. Further, when a medicine is combined with another, the combination may lead to a different set of side effects or accumulated side effects that would have not been caused by their consumption individually. For example, a first drug may be the best choice for treatment of a certain disease, yet it may cause adverse side effects that cause another symptom which is treated with yet another drug that may cause another set of side effects and so on. However, there may not be a suitable substitute for the first drug that reduces the severity or eliminates the side effects altogether and thus stops the buildup of complex medication there still may be another way to use the drug for example lower dosage to control the side effects. If this is known in advance, the medical treatment prescribed by the health care practitioner can be accordingly modified to allow for the first drug to be continued.

Furthermore, as the number of combinations of drugs increases, it becomes increasingly challenging if not impossible for the health care practitioner to accurately determine the root cause of a side effect and make differential diagnostics between side effects and symptoms of actual disease particularly with reference to which combination of drugs causes which side effect. The side effects caused by a particular combination of medicines may also differ from one patient to another. Due to the level of complexity involved, there is a high probability that a combination of drugs prescribed based on a mental estimate by a healthcare provider may lead to unforeseen side effects, which could have disastrous consequences for patients.

Therefore, in light of the foregoing discussion, there exists a need to overcome the aforementioned drawbacks in existing approaches used by the health care practitioners to prescribe a combination of drugs.

### SUMMARY

The present disclosure provides a method of determining a prescription of two or more medicines for a patient, using a machine learning model executed on a server, wherein the method comprises:

obtaining a medical record of a new patient, wherein the medical record comprises at least one of medical history, diagnoses by a medical expert, a gender, an age or genome mapping of the new patient;

generating a medicine intake matrix by obtaining medicine intake data of two or more medicines in use by the new patient, wherein the medicine intake matrix comprises rows that represent medicines and columns that represent time units when the medicine intake data is measured, and each cell in the medicine intake matrix represents a quantity of medicine used during a time unit, wherein the two or more medicines in use are prescribed by a medical professional for the new patient;

generating a symptom level matrix by obtaining levels of symptoms associated with the two or more medicines from the new patient after intake, wherein the symptom level matrix comprises rows that represent symptoms, columns that represent time units, and each cell in the symptom level matrix represents a level of severity of a symptom;

determining, using the machine learning model, a transfer function (F), based on the medicine intake matrix and the symptom level matrix; and;

determining, using the transfer function, a prescription comprising an amount of a first medicine and an amount of a second medicine for the new patient to reduce a value of a sum of the symptom level matrix, wherein the transfer function provides an output value of prescription for the new patient based on the medicine intake matrix and the symptom level matrix.

It will be appreciated that the aforesaid present method has a technical effect in that the method functions as a form of technical control using machine learning of a technical artificially intelligent system. The method involves building an artificially intelligent machine learning model and/or using the machine learning model to solve the technical problem of determining a prescription that comprises one or more medicines for a particular patient while minimizing side effects that could arise due to the patient, individual medicines, and/or combinations of medicines by using the machine learning model, a medicine intake matrix, a symptom level matrix and a transfer function.

The present disclosure also provides a system comprising a server for determining a prescription of two or more medicines for a patient, using a machine learning model, comprising:

a first processor; and

a memory configured to store program codes comprising:
a medical record obtaining module implemented by the first processor configured to obtain a medical record of a new patient, wherein the medical record comprises at least one of medical history, diagnoses by a medical expert, a gender, an age or genome mapping of the new patient;
a medicine intake matrix generation module implemented by the first processor configured to generate a medicine intake matrix by obtaining medicine intake data of the two or more medicines in use by the new patient, wherein the medicine intake matrix comprises rows that represent medicines and columns that represent time units when the medicine intake data is measured, and each cell in the medicine intake matrix represents a quantity of medicine used during a time unit, wherein the two or more medicines in use are prescribed by a medical professional for the new patient;
a symptom level matrix generation module implemented by the first processor configured to generate a symptom level matrix by obtaining levels of symptoms associated with the two or more medicines from the new patient after intake, wherein the symptom level matrix comprises rows that represent symptoms, columns that represent time units, and each cell in the symptom level matrix represents a level of severity of a symptom;
a transfer function determination module implemented by the first processor configured to determine, using the machine learning model, a transfer function (F), based on the medicine intake matrix and symptom level matrix, wherein the machine learning model is generated by a second processor configured to
   generate a first database with medicine data and associated symptoms of treated patients, wherein the medicine data comprises medicines that are prescribed to the treated patients and an amount of prescribed medicines that are consumed by the treated patients,
   generate a second database with medical records of the treated patients, wherein the medical records comprise at least one of medical history, diagnoses by a medical expert, gender, age or genome mapping of the treated patients,
   process an expert input from a medical expert on the medicine data of the treated patients, wherein the expert input comprises feedback associated with the medicine data of the treated patients, and
   provide the medicine data, the associated symptoms, the expert input on the medicine data and medical records of the treated patients to a machine learning algorithm as training data to generate the machine learning model; and
a prescription determination module implemented by the first processor configured to determine a prescription comprising a quantity of a first medicine and a quantity of a second medicine for the new patient to reduce a value of a sum of symptom level matrix using the transfer function.

The present disclosure also provides a method of generating a machine learning model to determine a prescription of two or more medicines for a patient, using a machine learning algorithm executed on a server, characterized in that the method comprises:

generating a first database with medicine data and associated symptoms of treated patients, wherein the medicine data comprises medicines that are prescribed to the treated patients and an amount of prescribed medicines that are consumed by the treated patients;

generating a second database with medical records of the treated patients, wherein the medical records comprise at least one of medical history, diagnoses by a medical expert, gender, age or genome mapping of the treated patients;

processing an expert input from a medical expert on the medicine data of the treated patients, wherein the expert input comprises feedback associated with the medicine data of the treated patients; and

providing the medicine data, the associated symptoms, the expert input on the medicine data and the medical records of the treated patients to the machine learning algorithm as training data to generate the machine learning model.

Embodiments of the present disclosure substantially eliminate or at least partially address the aforementioned drawbacks in existing approaches used by the health care practitioner to prescribe a combination of drugs.

Additional aspects, advantages, features and objects of the present disclosure are made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIG. 1 is a schematic illustration of a system in accordance with an embodiment of the present disclosure;

FIG. 2 is a schematic illustration of a system comprising a second processor that generates a machine learning model in accordance with an embodiment of the present disclosure;

FIG. 3 is a functional block diagram of a server in accordance with an embodiment of the present disclosure;

FIG. 4 is an exemplary tabular view of a first database in accordance with an embodiment of the present disclosure;

FIG. 5 is an exemplary tabular view of a second database in accordance with an embodiment of the present disclosure;

FIG. 6 is an exemplary view of a graphical user interface of a user device in accordance with an embodiment of the present disclosure;

FIG. 7 is an exemplary view of a graphical user interface of a user device in accordance with an embodiment of the present disclosure;

FIG. 8 is an exemplary view of a heatmap that is generated by a server based on medicine intake data of a patient in accordance with an embodiment of the present disclosure;

FIG. 9 is an exemplary view of a heatmap that is generated by a server based on levels of symptoms associated with two or more medicines after intake by a patient in accordance with an embodiment of the present disclosure;

FIG. 10 is an exemplary view of a heatmap that is generated by a server based on a level of symptom associated with a medicine after intake by a patient in accordance with an embodiment of the present disclosure;

FIG. 11 is an exemplary view of a heatmap that is generated by a server based on medicine intake data of a patient in accordance with an embodiment of the present disclosure;

FIG. 12 is an exemplary view of a heatmap that is generated by a server based on levels of symptoms associated with medicines after intake by a patient in accordance with an embodiment of the present disclosure; and

FIGS. 13A and 13B are flow diagrams illustrating a method of determining a prescription of two or more medicines for a patient in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

The present disclosure provides a method of determining a prescription of two or more medicines for a patient, using a machine learning model executed on a server, wherein the method comprises:

obtaining a medical record of a new patient, wherein the medical record comprises at least one of medical history, diagnoses by a medical expert, a gender, an age or genome mapping of the new patient;

generating a medicine intake matrix by obtaining medicine intake data of two or more medicines in use by the new patient, wherein the medicine intake matrix comprises rows that represent medicines and columns that represent time units when the medicine intake data is measured, and each cell in the medicine intake matrix represents a quantity of medicine used during a time unit, wherein the two or more medicines in use are prescribed by a medical professional for the new patient;

generating a symptom level matrix by obtaining levels of symptoms associated with the two or more medicines from the new patient after intake, wherein the symptom level matrix comprises rows that represent symptoms, columns that represent time units, and each cell in the symptom level matrix represents a level of severity of a symptom;

determining, using the machine learning model, a transfer function (F), based on the medicine intake matrix and the symptom level matrix; and

determining, using the transfer function, a prescription comprising an amount of a first medicine and an amount of a second medicine for the new patient to reduce a value of a sum of the symptom level matrix, wherein the transfer function provides an output value of prescription for the new patient based on the medicine intake matrix and the symptom level matrix.

The present method thus helps to determine a suitable prescription comprising a combination of the medicines (e.g. the amount of the first medicine and the amount of the second medicine) for the new patient based on the analysis of medicine data of the treated patients, symptoms associated with the medicine data and an expert input on the medicine data. The present method thus helps to modify the combination or a composition of the two or more medicines based on a feedback on the levels of symptoms from the patient. The present method also simulates likely results and/or symptoms to occur due to modification of the combination or the composition of the two or more medicines in use. Further advantage of the method is that quantity of used medicine can be reduced.

It will be appreciated that the aforesaid present method is not merely a “method of doing a mental act”, but has technical effect in that the method functions as a form of technical control using machine learning of a technical artificially intelligent system. The method involves building an artificially intelligent machine learning model and/or using the machine learning model to solve the technical problem of determining a prescription that comprises one or more medicines for a particular patient while minimizing side effects that could arise due to the patient, individual medicines, and/or combinations of medicines by using the machine learning model, a medicine intake matrix, a symptom level matrix and a transfer function.

The present method may provide the prescription comprising the amount of the first medicine and the amount of the second medicine of the new patient as training data to train the machine learning model. The present method may perform differential diagnostics automatically to differentiate the symptoms caused by the two or more medicines in use and symptoms caused by a disease that the patient suffers from. The medical professional may be a doctor, a nurse, a lab technician, a clinician or a care taker. In an embodiment, the medical record of the new patient is obtained from a user device of the new patient. In another embodiment, the medical record of the new patient is obtained from an expert device of the medical professional. The machine learning model may be defined as a model artifact that is created by a training process.

The medicine intake data may be obtained from the user device of the new patient. The user device may provide a first graphical user interface to the new patient to input the medicine intake data. The medicine intake data comprises an amount of the two or more medicines that are consumed by the new patient. The levels of symptoms associated with the two or more medicines in use may be obtained from the user device of the new patient. The user device may provide a second graphical user interface to the new patient to input the levels of symptoms caused by the two or more medicines after intake. The levels of symptoms associated with the two or more medicines in use may be obtained automatically by a biosensor. The biosensor may comprise a heart rate monitor, an Electroencephalogram (EEG), an Electrocardiogram (ECG or EKG) or a thermometer. The transfer function (F) may be determined by the following equation:

A×F=B

F=A′×B

Where A is the medicine intake matrix, B is the symptom level matrix, A′ is an inverse of the medicine intake matrix and F is the transfer function. In an embodiment, the medicine intake matrix and the symptom level matrix are dimensioned in a way, such that no exact solution for the transfer function is determined. As an example values in the symptom level matrix B row one might be a time series representation of a measurement data from a first biosensor and values in the symptom level matrix B row two might be a time series representation of a measurement date from a second biosensor etc. Hence, the machine learning model may be applied to determine the transfer function.

According to an embodiment, the machine learning model is generated by

generating a first database with medicine data and associated symptoms of treated patients, wherein the medicine data comprises medicines that are prescribed to the treated patients and an amount of prescribed medicines that are consumed by the treated patients;

generating a second database with medical records of the treated patients, wherein the medical records comprise at least one of medical history, diagnoses by a medical expert, gender, age or genome mapping of the treated patients;

processing an expert input from a medical expert on the medicine data of the treated patients, wherein the expert input comprises feedback associated with the medicine data of the treated patients; and

providing the medicine data, the associated symptoms, the expert input on the medicine data and the medical records of the treated patients to a machine learning algorithm as training data to generate the machine learning model.

The machine learning model may be generated by the server. The first database and/or the second database may be generated by the server. The expert input may be obtained from an expert device associated with the medical expert.

According to another embodiment, method comprises grouping, using the machine learning model, two or more patients of the same type from the treated patients based on at least one of the gender, the age or the genome mapping of the new patient.

According to yet another embodiment, the method comprises using the machine learning model to generate a recommendation on the prescription for the new patient. In an embodiment, the recommendation on the prescription is selected from a group comprising at least one of keeping a current prescription of the two or more medicines in use, reducing an amount of at least one medicine among the two or more medicines with x percentage, increasing an amount of at least one medicine among the two or more medicines with y percentage, removing at least one medicine from the two or more medicines or adding a new medicine to the two or more medicines. The recommendation on the prescription may be used to train a neural network of the machine learning algorithm.

According to yet another embodiment, the method comprises obtaining a composition data input comprising a composition of the two or more medicines in use that are prescribed by the medical professional for the new patient.

According to yet another embodiment, the method comprises obtaining a score for each level of symptoms associated with the two or more medicines from the new patient after intake. The score may be obtained manually from the new patient through the user device for each level of symptoms associated with the two or more medicines in use.

Alternatively or additionally the score may be obtained using output values of one or more biosensors automatically. As an example if a thermometer data shows decrease of body temperature to normal level a score indication that a symptoms are positive. As another example if a heart rate monitor shows problems with a heart after medicine intake the score indication is that symptoms are negative. As a further example biosensor data can be arranged in time series. In such a case score can be based on for example sliding average or rate of change. According to an embodiment the levels of symptoms for the symptom level matrix are obtained from one or more biosensors.

According to yet another embodiment, the method comprises using the machine learning model to provide information on symptoms to follow for the prescription after intake by the new patient. In an embodiment, the machine learning model obtains information on rating of the symptoms that occurred after intake of the prescribed medicine over a period of time.

According to yet another embodiment, the method comprises using the machine learning model to differentiate automatically the symptoms being caused by the two or more medicines in use after intake from symptoms of a disease that the new patient suffers from. The machine learning model may also determine whether the symptom is caused due to addition of at least one new medicine to the two or more medicines in use or an over dosage of at least one medicine among the two or more medicines in use.

The present disclosure provides a system comprising a server for determining a prescription of two or more medicines for a patient, using a machine learning model, comprising:

a first processor; and

a memory configured to store program codes comprising:
a medical record obtaining module implemented by the first processor configured to obtain a medical record of a new patient, wherein the medical record comprises at least one of medical history, diagnoses by a medical expert, a gender, an age or genome mapping of the new patient;
a medicine intake matrix generation module implemented by the first processor configured to generate a medicine intake matrix by obtaining medicine intake data of the two or more medicines in use by the new patient, wherein the medicine intake matrix comprises rows that represent medicines and columns that represent time units when the medicine intake data is measured, and each cell in the medicine intake matrix represents a quantity of medicine used during a time unit, wherein the two or more medicines in use are prescribed by a medical professional for the new patient;
a symptom level matrix generation module implemented by the first processor configured to generate a symptom level matrix by obtaining levels of symptoms associated with the two or more medicines from the new patient after intake, wherein the symptom level matrix comprises rows that represent symptoms, columns that represent time units, and each cell in the symptom level matrix represents a level of severity of a symptom;
a transfer function determination module implemented by the first processor configured to determine, using the machine learning model, a transfer function (F), based on the medicine intake matrix and symptom level matrix, wherein the machine learning model is generated by a second processor configured to
   generate a first database with medicine data and associated symptoms of treated patients, wherein the medicine data comprises medicines that are prescribed to the treated patients and an amount of prescribed medicines that are consumed by the treated patients,
   generate a second database with medical records of the treated patients, wherein the medical records comprise at least one of medical history, diagnoses by a medical expert, gender, age or genome mapping of the treated patients,
   process an expert input from a medical expert on the medicine data of the treated patients, wherein the expert input comprises feedback associated with the medicine data of the treated patients, and
   provide the medicine data, the associated symptoms, the expert input on the medicine data and medical records of the treated patients to a machine learning algorithm as training data to generate the machine learning model; and
a prescription determination module implemented by the first processor configured to determine a prescription comprising a quantity of a first medicine and a quantity of a second medicine for the new patient to reduce a value of a sum of symptom level matrix using the transfer function.

The advantages of the present system are thus identical to those disclosed above in connection with the present method and the embodiments listed above in connection with the method apply mutatis mutandis to the system.

The medicine data may be obtained from the treated patients using a first input means. The symptoms associated with the medicine data may be obtained using a second input means. In an embodiment, the first input means and the second input means are communicatively connected to the server over a communication network. The first input means or the second input means may comprise a personal computer, a smart phone, a tablet, a laptop or an electronic notebook. The communication network may be a wired network or a wireless network. The server may be a tablet, a desktop, a personal computer or an electronic notebook. In an embodiment, the server may be a cloud service.

The server may partially comprise the above modules to determine the prescription of the two or more medicines for the new patient. The system may comprise more than one server that may comprise one or more of the above modules. In an embodiment, the server comprises the second processor. The second processor may execute the one or more of the above modules. In another embodiment, the second processor is executed in an external server. The machine learning model may be generated by the first processor. The server may comprise a server database that stores the machine learning model.

In an embodiment the symptom level matrix generation module is configured to receive data from one or more biosensors. As an example a live data feed from a biosensor is received by the symptom level matrix generator. Alternatively a set of measured biosensor data is received by the symptom level matrix generator from time to time. The symptom level matrix generator is configured to arrange received data to form a matrix.

According to an embodiment, the system comprises

a patient grouping module implemented by the first processor configured to group two or more patients of the same type from the treated patients based on at least one of the gender, the age or the genome mapping of the new patient using the machine learning model; and

a recommendation module implemented by the first processor configured to generate a recommendation on the prescription for the new patient.

In an embodiment, the recommendation module is implemented by the second processor. According to another embodiment, the system comprises a user device, communicatively connected to the server, for reporting at least one of the medicine intake data or the levels of symptoms associated with the two or more medicines by the new patient after intake. The user device may be a mobile phone, a personal computer, a laptop, a Smartphone or an electronic notebook. In an embodiment, the user device is communicatively connected to the server through a communication network. The server may be a personal computer, a mobile phone, a laptop, a Smartphone or an electronic notebook. In an embodiment, the new patient reports manually, using the user device, at least one of the medicine intake data or the levels of symptoms associated with the two or more medicines in use to the server. In alternative or additional embodiment the one or more biosensors are used to determine levels of symptoms or biosensor data is used to determine levels of symptoms.

According to yet another embodiment, the system comprises an expert device, communicatively connected to the server, for monitoring the reporting by the new patient after intake of the two or more medicine and usage of the two or more medicines as per medical professional prescription, wherein the expert device comprises a user interface that enables the medical professional to provide an expert input on the medicine intake data and the symptoms associated with the two or more medicines. In an embodiment, the expert device is communicatively connected to the server through a communication network. The expert device may be a personal computer, a mobile phone, a laptop, a Smartphone or an electronic notebook. The medical professional may be a doctor, a nurse, a care taker, a lab technician or a clinician.

In an embodiment the levels of symptoms are derived from data received from one or more biosensors. One or more biosensors might be communicatively coupled to the server in real time manner or those might provide data from time to time. Data might be preprocessed (to indicate symptom) or might be raw data. In latter case analysis of raw data provides the level of symptom. The data can be in time series.

The present disclosure also provides a method of generating a machine learning model to determine a prescription of two or more medicines for a patient, using a machine learning algorithm executed on a server, characterized in that the method comprises:

generating a first database with medicine data and associated symptoms of treated patients, wherein the medicine data comprises medicines that are prescribed to the treated patients and an amount of prescribed medicines that are consumed by the treated patients;

generating a second database with medical records of the treated patients, wherein the medical records comprise at least one of medical history, diagnoses by a medical expert, gender, age or genome mapping of the treated patients;

processing an expert input from a medical expert on the medicine data of the treated patients, wherein the expert input comprises feedback associated with the medicine data of the treated patients; and

providing the medicine data, the associated symptoms, the expert input on the medicine data and the medical records of the treated patients to the machine learning algorithm as training data to generate the machine learning model.

According to an embodiment, the method comprises

obtaining a medical record of a new patient, wherein the medical record comprises at least one of medical history, diagnoses by a medical expert, a gender, an age or genome mapping of the new patient;

using the machine learning model to group two or more patients of the same type from the treated patients based on at least one of the gender, the age or the genome mapping of the new patient;

generating a medicine intake matrix by obtaining medicine intake data of two or more medicines in use by the new patient, wherein the medicine intake matrix comprises rows that represent medicines and columns that represent time units when the medicine intake data is measured, and each cell in the medicine intake matrix represents a quantity of medicine used during a time unit, wherein the two or more medicines in use are prescribed by a medical professional for the new patient;

generating a symptom level matrix by obtaining levels of symptoms associated with the two or more medicines from the new patient after intake, wherein the symptom level matrix comprises rows that represent symptoms, columns that represent time units, and each cell in the symptom level matrix represents a level of severity of a symptom;

using the machine learning model to determine a transfer function (F), based on the medicine intake matrix and the symptom level matrix; and

determining, using the transfer function, a prescription comprising an amount of a first medicine and an amount of a second medicine for the new patient to reduce a value of a sum of the symptom level matrix, wherein the transfer function provides an output value of prescription for the new patient based on the medicine intake matrix and the symptom level matrix.

In an embodiment the system level matrix is generated using a time series based biosensor data. Time series refers to having multiple measurement points over time. For example measurement can be done every second, minute, once a day etc. When generating the matrix the measurement data can be interpolated to have common time line.

The advantages of this method are thus identical to those disclosed above in connection with the method of determining the prescription of two or more medicines for the patient as described above and the embodiments listed above in connection with the method of determining the prescription of two or more medicines for the patient as described above apply mutatis mutandis to the present method.

### Experiments/Examples of Benefits

In a first example, a schizophrenic patient suffers from intense motoric disorder. The symptoms associated with the intense motoric disorder may comprise severe tremor in hands of the schizophrenic patient. The symptoms may also comprise an appearance of bending the knees in exaggerated manner by the schizophrenic patient when walking. The schizophrenic patient suffers from hallucinations when he/she consumes an over dosage of neuroleptic medication. Further, the schizophrenic patient suffers from agitation due to the over dosage of the neuroleptic medication. The neuroleptic medication that is prescribed to the schizophrenic patient may comprise aspirin (ASA), olanzapine, multivitamine, Valproate and latanoprost/timolol. In an example the symptoms can be monitored with a biosensor such as a movement sensor (having accelerometer) attached to the patient. Accelerometer readings can be used as an input to the system to determine the motoric disorder in given example.

The present method may identify symptoms caused by olanzapine and valproate and generate a recommendation to reduce valproate using the machine learning model. The machine learning model, from medicine data of the schizophrenic patient, may identify that a dosage of olanzapine is increased whenever a dosage of valproate is increased due to worsening mental disease symptoms. The machine learning model may identify that the worsening mental disease symptoms is caused due to the over dosage of valproate, and not due to schizophrenia. The machine learning model may perform differential diagnostics to differentiate the worsening symptoms caused by the valproate and symptoms caused by the schizophrenia based on the medicine data of the schizophrenic patient.

After identifying that the worsening symptoms is caused by the over dosage of valproate, the machine learning model may generate a recommendation to reduce the dosage of valproate and the dosage of olanzapine gradually for the schizophrenic patient for a couple of cycles to reduce the worsening symptoms. After reducing the dosage of valproate and the dosage of olanzapine for each cycle, the machine learning model may provide information on subsequent symptoms to follow after intake of reduced dosage of valproate and olanzapine by the schizophrenic patient to the medical professionals and may obtain information on rating on the subsequent symptoms after a period of time (e.g. two weeks). After the couple of reduction cycles, the machine learning model may determine an optimal dosage of valproate (e.g. valproate—100 milligrams (mg) 1×3 (i.e. one pill three times a day)) and olanzapine (e.g. Olanzapine—5 mg ½×1 (half pill once a day)) to be prescribed for the schizophrenic patient. In given example feedback loop to the machine learning system could be the accelerometer reading of the biosensor.

In second example, an elderly woman patient suffers from unidentified dementia, and is being medicated with an antipsychotic drug named risperidone. Typically, risperidone is prescribed for behavioral disturbances. Further, the woman patient suffers from symptoms such as apathetic, rigid and dystonia when she is lying in on bed and leaving her feet hanging over an edge of the bed. As the feet of the woman patient hanging over the edge of the bed, the woman patient finds difficulty in sleeping, and thus leads to sleeping disorder. Therefore, a small dosage of mirtazapine is prescribed to the woman patient for the sleeping disorder.

The present method, using the machine learning model, may identify that the apathy, rigidity and dystonia are side effects of risperidone. After identifying that the above symptoms are caused due to the over dosage of risperidone, the machine learning model may generate a recommendation to reduce a dosage of risperidone for the woman patient for a couple of cycles in order to reduce a severity of the side effects. The machine learning model may provide information on subsequent symptoms to follow after intake of the reduced dosage of risperidone by the women patient to the medical professional. After reducing the dosage of risperidone for couple of cycles, the woman patient was not suffering from apathy, rigidity and dystonia as before and her feet are started to stay wholly on the bed while sleeping, thus easing the sleeping disorder.

After reducing the dosage of the risperidone for the couple of cycles, the machine learning model may generate a recommendation to reduce the dosage of mirtazapine, which is prescribed for the sleeping disorder, for a couple of cycles. The machine learning model may determine whether the mirtazapine is needed for the woman patient as the dosage of the risperidone which causes the sleeping disorder has been reduced. After reducing the dosage of the mirtazapine for couple of cycles, the woman patient starts sleeping well as before. Here, the machine learning model performs differential diagnostics to differentiate the sleeping disorder caused by the risperidone and an actual sleeping disorder and determines an optimal dosage of risperidone (e.g. risperidone—0.5 mg 1×2 (one pill two times a day)) for treating the women patient.

In third example embodiment, an elderly woman patient is diagnosed with high blood pressure, Alzheimers' disease and heart failure. The woman patient suffers from severe swelling in her lower extremity focusing around her ankles. The woman patient also suffers from alarmingly low blood pressure (e.g. systolic pressure) which is above hundred. The woman patient is being prescribed with Apixabane 5 milligrams (mg), Felodipine 2.5 mg 2+1 (two pills once a day and one pill once a day), Furosemide 40 mg 1×2 (one pill two times a day), Galantamine 24 mg 1×1 (one pill per day), Potassium 1 g 1×1 (one pill per day), Losartane 1×1 (one pill per day), Omeprazole 20 mg 1×1 (one pill per day), and Parasetamol 500 mg 2×3 (two pills three times a day) for treating the high blood pressure, the Alzheimers' disease and the heart failure. The present method, using the machine learning model, may identify that swelling in her lower extremity is a common side effect due to the dosage of felodipine. The machine learning model may generate a recommendation to reduce a dosage of felodipine for a couple of cycles for reducing the swelling in her ankles, which is diagnosed as a heart failure. The machine learning model may provide information on subsequent symptoms to follow after intake of the reduced dosage of felodipine by the women patient to the medical professional. After reducing the dosage of the felodipine for couple of cycles, the elderly woman is not suffering from ankles swelling and thus dismantling the diagnoses of the heart failure. As the diagnoses of the heart failure is dismantled, the machine learning model may generate a recommendation for the woman patient to remove the dosage of Furosemide that is prescribed for the woman patient for the heart failure diagnose. Typically, the furosemide has a side effect of reducing the potassium level in the woman patient after intake. In order to maintain the potassium level, the potassium is prescribed as a medicine by the medical professional for the woman patient. As the furosemide is removed from the prescription, the machine learning model may generate a recommendation to remove the dosage of potassium from the prescription. The machine learning model performs differential diagnostics to differentiate the symptoms caused by the felodipine and actual disease symptoms and determine an optimal prescription for the woman patient as Apixabane 5 mg, Galantamine 24 mg 1×1 (one pill per day), Losartane 1×1 (one pill per day), Omeprazole 20 mg 1×1 (one pill per day) and Parasetamol 500 mg 2×3 (two pills per three times a day). In given example a biosensor of heart pressure monitoring system might be used to provide data to the system. Also biosensor such as smart clothing providing information of swelling can be used.

Embodiments of the present disclosure may determine a suitable prescription comprising a combination of the medicines (e.g. an amount of first medicine and an amount of second medicine) for the new patient. Embodiments of the present disclosure may modify a combination or a composition of the two or more medicines based on a feedback on the levels of symptoms from the new patient. The embodiments of the present disclosure may determine which of the medicine impacts on which of the patient using the machine learning model. The embodiments of the present disclosure may train the system to generate a recommendation on the prescription for the new patient based on the analysis of the medicine data of the treated patients, the symptoms associated with the medicine data and an expert input on the medicine data. Embodiments of the present disclosure may eliminate the limitations in determining prescription for the new patient and identifying symptoms caused by a combination of two or medicines after intake by the new patient.

### DETAILED DESCRIPTION OF THE DRAWINGS

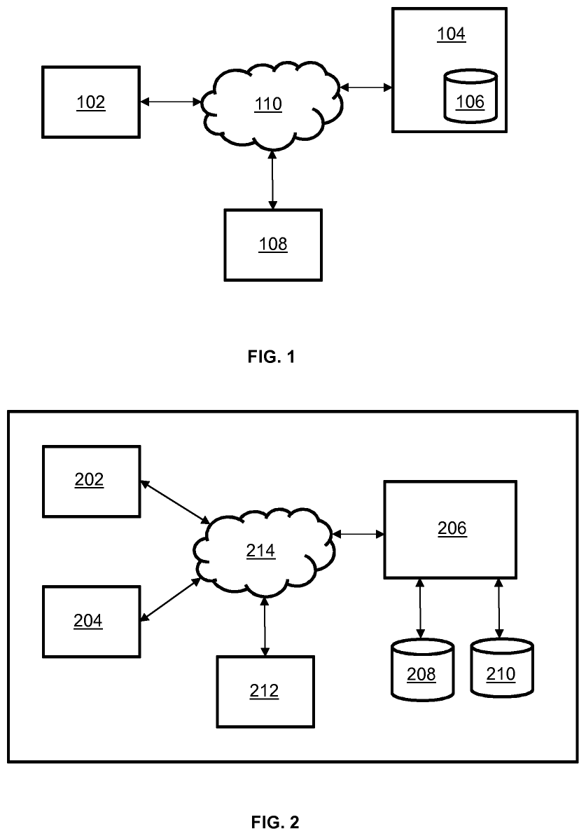

FIG. 1 is a schematic illustration of a system in accordance with an embodiment of the present disclosure. The system comprises a user device 102, a server 104, an expert device 108 and a communication network 110. The server 104 comprises a first processor and a server database 106. The function of these parts as has been described above.

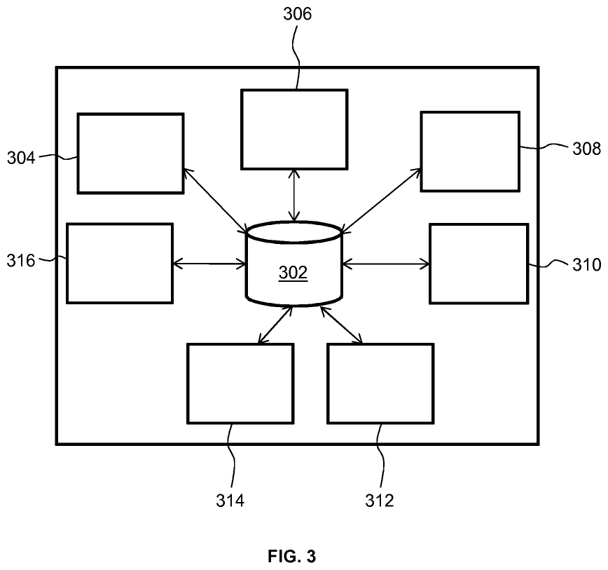

FIG. 2 is a schematic illustration of a system comprising a second processor 206 that generates a machine learning model in accordance with an embodiment of the present disclosure. The system comprises a first input means 202, a second input means 204, the second processor 206, a first database 208, a second database 210, an expert device 212 and a communication network 214. The function of these parts as has been described above.

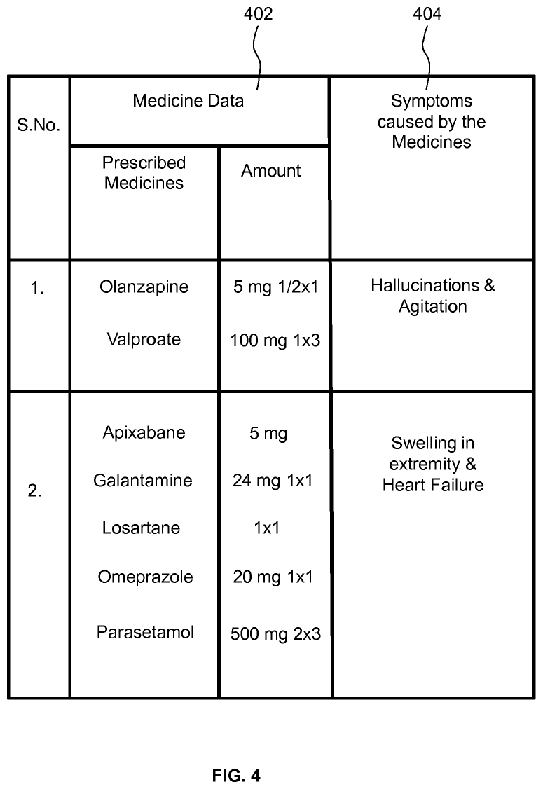

FIG. 3 is a functional block diagram of a server in accordance with an embodiment of the present disclosure. The functional block diagram of the server comprises a server database 302, a medical record obtaining module 304, a patient grouping module 306, a medicine intake matrix generation module 308, a symptom level matrix generation module 310, a transfer function determination module 312, a prescription determination module 314 and a recommendation module 316. These modules function as has been described above.

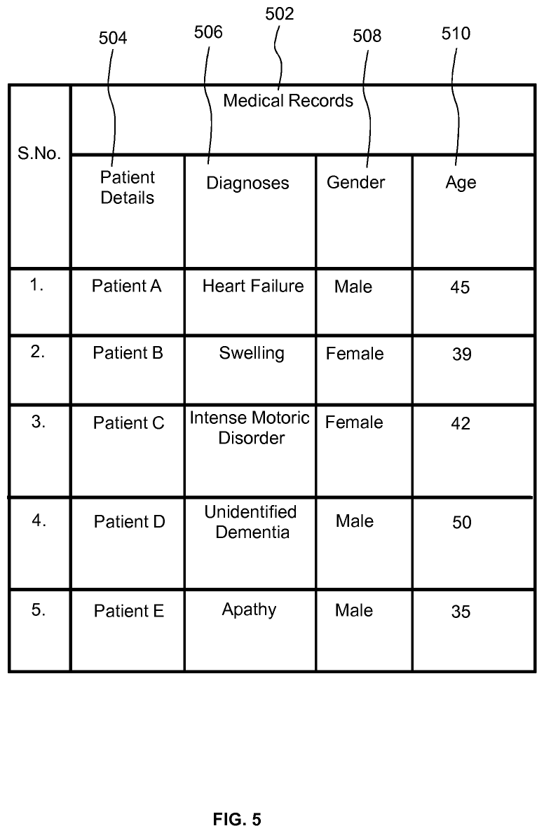

FIG. 4 is an exemplary tabular view of a first database in accordance with an embodiment of the present disclosure. The tabular view comprises a medicine data field 402 and a symptoms field 404. The medicine data field 402 comprises medicines that are prescribed to treated patients and an amount of prescribed medicines that are consumed by the treated patients. The symptoms field 404 comprises associated symptoms that are caused due to consumption of the prescribed medicines by the treated patients.

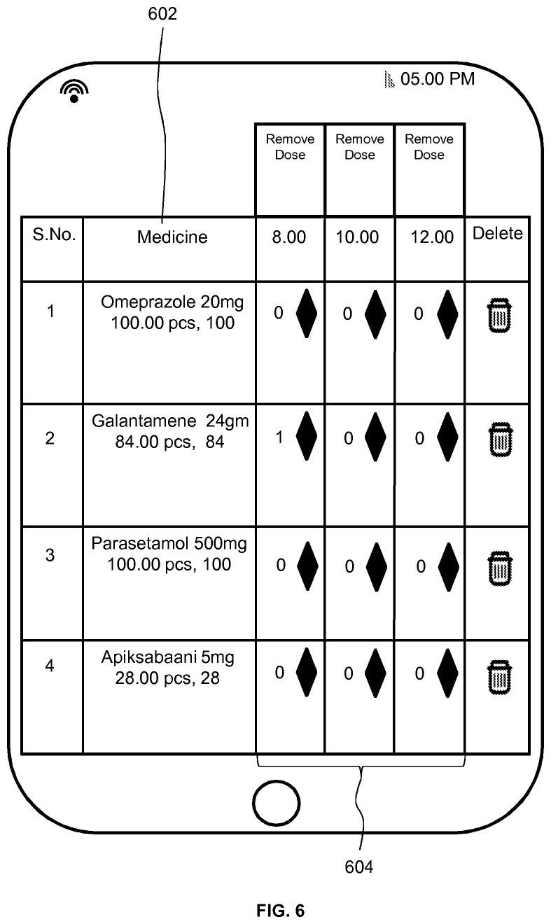

FIG. 5 is an exemplary tabular view of a second database in accordance with an embodiment of the present disclosure. The tabular view comprises a medical records field 502. The medical records field 502 comprises a patient details field 504, a diagnoses field 506, a gender field 508 and an age field 510. The patient details field 504 may comprise details of treated patients. The diagnoses field 506 may comprise details of diseases that the treated patients suffer from. The gender field 508 may comprise a gender of the treated patients. The age field 510 may comprise an age of the treated patients. The tabular view may further comprise a genome mapping field. The genome mapping field may comprise genome mapping of the treated patients.

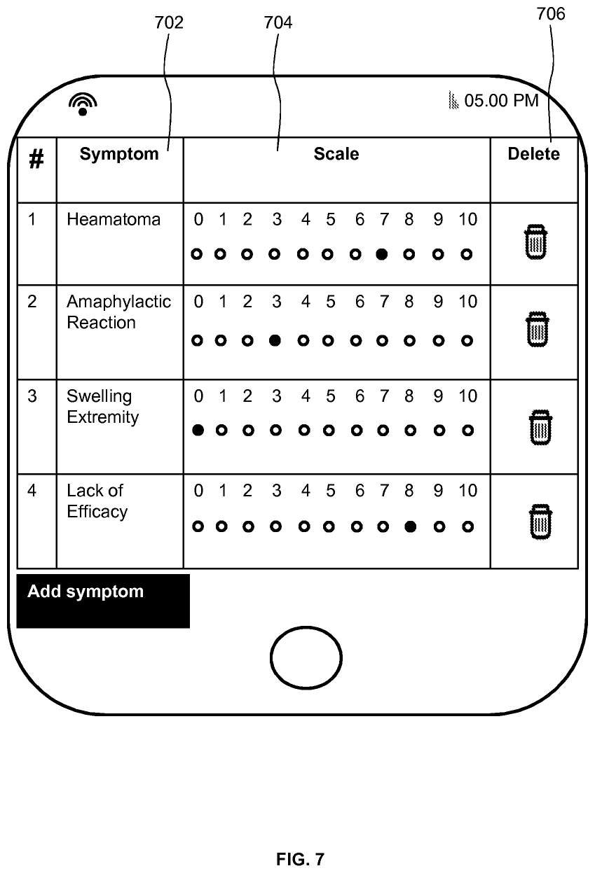

FIG. 6 is an exemplary view of a graphical user interface of a user device in accordance with an embodiment of the present disclosure. The graphical user interface comprises a medicine field 602 and a dosage field 604. The medicine field 602 may comprise a list of two or more medicines that are prescribed for a patient. The graphical user interface may provide an option to the patient to input an amount of the two or more medicines that are consumed by the patient (e.g. medicine intake data) during a time unit in the dosage field 604.

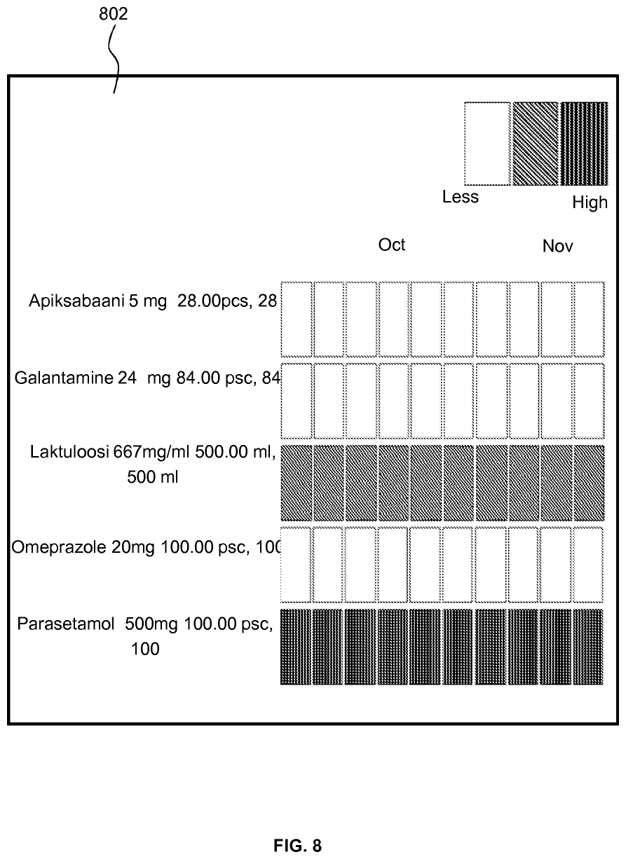

FIG. 7 is an exemplary view of a graphical user interface of a user device in accordance with an embodiment of the present disclosure. The graphical user interface comprises a symptom field 702, a scale field 704 and a delete field 706. The symptom field 702 may collect a list of symptoms that are being caused after intake of two or more medicines. The two or more medicines may be prescribed for a patient to treat a disease that he/she suffers from. The scale field 704 may provide an option to the patient to select a level of severity of a symptom that is being caused after intake of the two or more medicines by the patient. The delete field 706 may provide an option to the patient to delete a symptom from the list of symptoms.

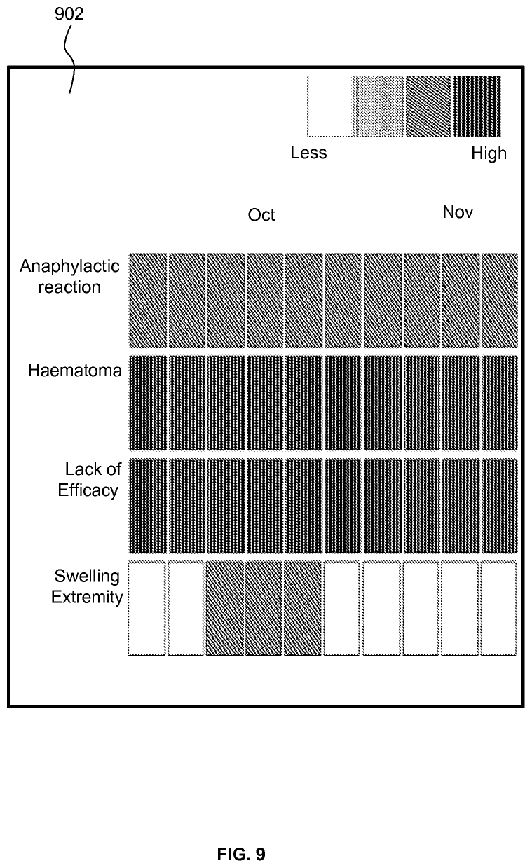

FIG. 8 is an exemplary view of a heatmap 802 that is generated by a server based on medicine intake data of a patient in accordance with an embodiment of the present disclosure. The medicine intake data may comprise an amount of two or more medicines (e.g. Apixabane, Galantamine, Lactulose, Omeprazol and Paracetamol) that are consumed by a patient. The two or more medicines may be prescribed for the patient to treat a disease. The heatmap 802 may show one or more cells (e.g. in a scale of 1-10) that correspond to an amount of the two or more medicines that are consumed by the patient during a time unit (e.g. October to November). For example, 1-10 cells may represent the amount of the two or more medicines that are consumed by the patient during different time units.

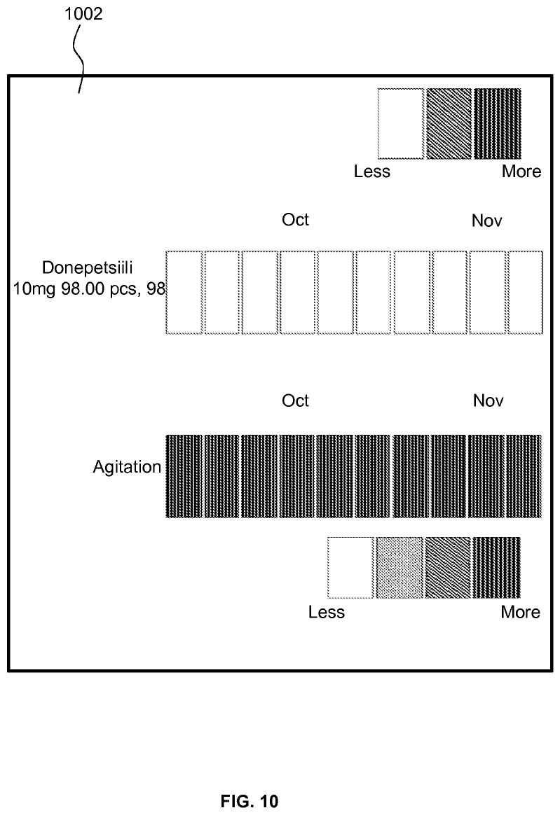

FIG. 9 is an exemplary view of a heatmap 902 that is generated by a server based on levels of symptoms associated with two or more medicines after intake by a patient in accordance with an embodiment of the present disclosure. The heatmap 902 may show one or more cells (e.g. in a scale of 1-10) that correspond to a level of a severity of symptoms associated with the two or more medicines after intake by the patient during a time unit (e.g. on daily basis from October to November).

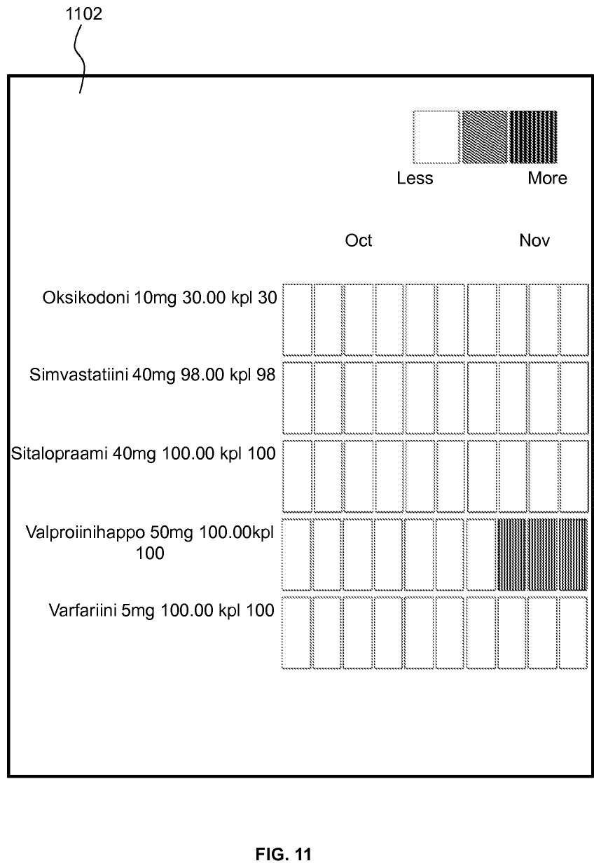

FIG. 10 is an exemplary view of a heatmap 1002 that is generated by a server based on a level of symptom associated with a medicine after intake by a patient in accordance with an embodiment of the present disclosure. The heatmap 1002 may show one or more first cells (e.g. in a scale of 1-10) that correspond to an amount of the medicine that is consumed by the patient during a time period (e.g. October to November). The heatmap 1002 further may show one or more second cells (e.g. in a scale of 1-10) that correspond to a level of severity of a symptom associated with the medicine during a time unit (e.g. on daily basis from October to November).

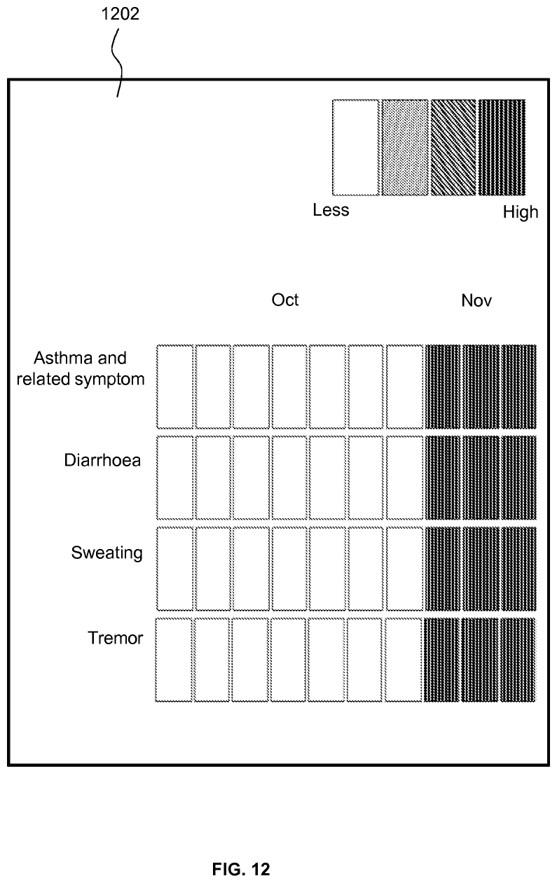

FIG. 11 is an exemplary view of a heatmap 1102 that is generated by a server based on medicine intake data of a patient in accordance with an embodiment of the present disclosure. The medicine intake data may comprise medicines such as Oxycodone, Simvastatine, Citalopram, Valproic acid and Warfarine that are prescribed for the patient. The above medicines may be prescribed for the patient to treat a disease. The heatmap 1102 may show one or more cells (e.g. in a scale of 1-10) that correspond to an amount of the above medicines that are consumed by the patient during a time unit (e.g. October to November).

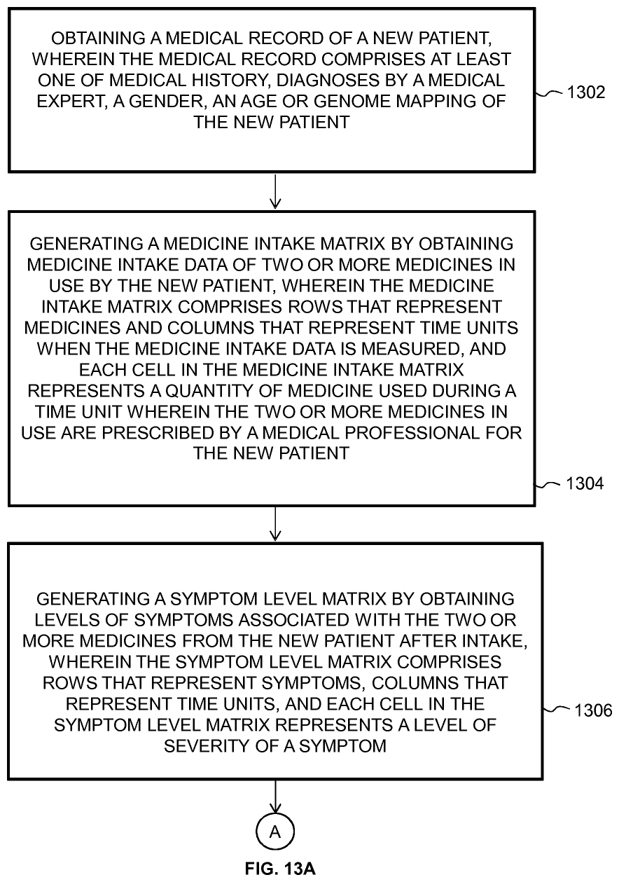

FIG. 12 is an exemplary view of a heatmap 1202 that is generated by a server based on levels of symptoms associated with medicines after intake by a patient in accordance with an embodiment of the present disclosure. The medicines may comprise Oxycodone, Simvastatine, Citalopram, Valproic acid and Warfarine. The heatmap 1202 may show one or more cells (e.g. in a scale of 1-10) that correspond to a level of severity of symptoms such as asthma and related symptom, Diarrhoea, Sweating and Tremor associated with the medicines during a time unit (e.g. on daily basis from October to November).

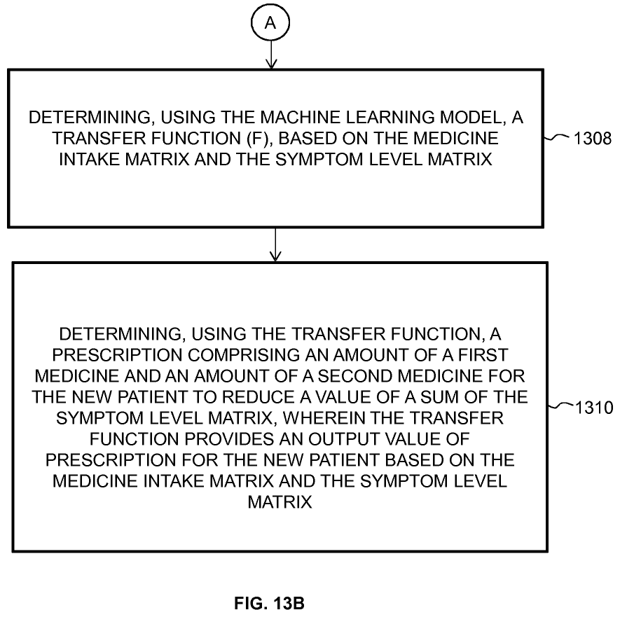

FIGS. 13A and 13B are flow diagrams illustrating a method of determining a prescription of two or more medicines for a patient in accordance with an embodiment of the present disclosure. At a step 1302, a medical record of a new patient is obtained. The medical record comprises at least one of medical history, diagnoses by a medical expert, a gender, an age or genome mapping of the new patient. At a step 1304, a medicine intake matrix is generated by obtaining medicine intake data of two or more medicines in use by the new patient. The medicine intake matrix comprises rows that represent medicines and columns that represent time units when the medicine intake data is measured, and each cell in the medicine intake matrix represents a quantity of medicine used during a time unit. The two or more medicines in use are prescribed by a medical professional for the new patient. At a step 1306, a symptom level matrix is generated by obtaining levels of symptoms associated with the two or more medicines from the new patient after intake. The symptom level matrix comprises rows that represent symptoms, columns that represent time units, and each cell in the symptom level matrix represents a level of severity of a symptom. At a step 1308, a transfer function is determined using the machine learning model based on the medicine intake matrix and the symptom level matrix. At a step 1310, a prescription comprising an amount of a first medicine and an amount of a second medicine for the new patient is determined to reduce a value of a sum of the symptom level matrix using the transfer function. The transfer function provides an output value of prescription for the new patient based on the medicine intake matrix and the symptom level matrix.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A method of determining a prescription of two or more medicines for a patient, using a machine learning model executed on a server, characterized in that the method comprises:
obtaining a medical record of a new patient, wherein the medical record comprises at least one of medical history, diagnoses by a medical expert, a gender, an age or genome mapping of the new patient;
generating a medicine intake matrix by obtaining medicine intake data of two or more medicines in use by the new patient, wherein the medicine intake matrix comprises rows that represent medicines and columns that represent time units when the medicine intake data is measured, and each cell in the medicine intake matrix represents a quantity of medicine used during a time unit, wherein the two or more medicines in use are prescribed by a medical professional for the new patient;
generating a symptom level matrix by obtaining levels of symptoms associated with the two or more medicines from the new patient after intake, wherein the symptom level matrix comprises rows that represent symptoms, columns that represent time units, and each cell in the symptom level matrix represents a level of severity of a symptom;
determining, using the machine learning model, a transfer function (F), based on the medicine intake matrix and the symptom level matrix; and
determining, using the transfer function, a prescription comprising an amount of a first medicine and an amount of a second medicine for the new patient to reduce a value of a sum of the symptom level matrix, wherein the transfer function provides an output value of prescription for the new patient based on the medicine intake matrix and the symptom level matrix.

2. A method according to claim 1, wherein the machine learning model is generated by
generating a first database with medicine data and associated symptoms of treated patients, wherein the medicine data comprises medicines that are prescribed to the treated patients and an amount of prescribed medicines that are consumed by the treated patients;
generating a second database with medical records of the treated patients, wherein the medical records comprise at least one of medical history, diagnoses by a medical expert, gender, age or genome mapping of the treated patients;
processing an expert input from a medical expert on the medicine data of the treated patients, wherein the expert input comprises feedback associated with the medicine data of the treated patients; and
providing the medicine data, the associated symptoms, the expert input on the medicine data and the medical records of the treated patients to a machine learning algorithm as training data to generate the machine learning model.

3. A method according to claim 1, wherein the method comprises grouping, using the machine learning model, two or more patients of the same type from the treated patients based on at least one of the gender, the age or the genome mapping of the new patient.

4. A method according to claim 1, wherein the method comprises using the machine learning model to generate a recommendation on the prescription for the new patient.

5. A method according to claim 1, wherein the method comprises obtaining a composition data input comprising a composition of the two or more medicines in use that are prescribed by the medical professional for the new patient.

6. A method according to claim 1, wherein the method comprises obtaining a score for each level of symptoms associated with the two or more medicines from the new patient after intake.

7. A method according to claim 1, wherein the method comprises using the machine learning model to provide information on symptoms to follow for the prescription after intake by the new patient.

8. A method according to claim 1, wherein the method comprises using the machine learning model to differentiate automatically the symptoms being caused by the two or more medicines in use after intake from symptoms of a disease that the new patient suffers from.

9. A method according to claim 1 wherein the levels of symptoms for the symptom level matrix are obtained from one or more biosensors.

10. A system comprising a server for determining a prescription of two or more medicines for a patient, using a machine learning model, comprising:
a first processor; and
a memory configured to store program codes comprising:
a medical record obtaining module implemented by the first processor configured to obtain a medical record of a new patient, wherein the medical record comprises at least one of medical history, diagnoses by a medical expert, a gender, an age or genome mapping of the new patient;
a medicine intake matrix generation module implemented by the first processor configured to generate a medicine intake matrix by obtaining medicine intake data of the two or more medicines in use by the new patient, wherein the medicine intake matrix comprises rows that represent medicines and columns that represent time units when the medicine intake data is measured, and each cell in the medicine intake matrix represents a quantity of medicine used during a time unit, wherein the two or more medicines in use are prescribed by a medical professional for the new patient;
a symptom level matrix generation module implemented by the first processor configured to generate a symptom level matrix by obtaining levels of symptoms associated with the two or more medicines from the new patient after intake, wherein the symptom level matrix comprises rows that represent symptoms, columns that represent time units, and each cell in the symptom level matrix represents a level of severity of a symptom;
a transfer function determination module implemented by the first processor configured to determine, using the machine learning model, a transfer function (F), based on the medicine intake matrix and symptom level matrix, wherein the machine learning model is generated by a second processor configured to
generate a first database with medicine data and associated symptoms of treated patients, wherein the medicine data comprises medicines that are prescribed to the treated patients and an amount of prescribed medicines that are consumed by the treated patients,
generate a second database with medical records of the treated patients, wherein the medical records comprise at least one of medical history, diagnoses by a medical expert, gender, age or genome mapping of the treated patients,
process an expert input from a medical expert on the medicine data of the treated patients, wherein the expert input comprises feedback associated with the medicine data of the treated patients, and
provide the medicine data, the associated symptoms, the expert input on the medicine data and medical records of the treated patients to a machine learning algorithm as training data to generate the machine learning model; and
a prescription determination module implemented by the first processor configured to determine a prescription comprising a quantity of a first medicine and a quantity of a second medicine for the new patient to reduce a value of a sum of symptom level matrix using the transfer function.

11. A system according to claim 10, wherein the system comprises
a patient grouping module implemented by the first processor configured to group two or more patients of the same type from the treated patients based on at least one of the gender, the age or the genome mapping of the new patient using the machine learning model; and
a recommendation module implemented by the first processor configured to generate a recommendation on the prescription for the new patient.

12. A system according to claim 10, wherein the system comprises a user device, communicatively connected to the server, for reporting at least one of the medicine intake data or the levels of symptoms associated with the two or more medicines by the new patient after intake.

13. A system according to claim 10, wherein the system comprises an expert device, communicatively connected to the server, for monitoring the reporting by the new patient after intake of the two or more medicine and usage of the two or more medicines as per medical professional prescription, wherein the expert device comprises a user interface that enables the medical professional to provide an expert input on the medicine intake data and the symptoms associated with the two or more medicines.

14. A system according to claim 10 wherein the levels of symptoms are derived from data received from one or more biosensors.

15. A method of generating a machine learning model to determine a prescription of two or more medicines for a patient, using a machine learning algorithm executed on a server, characterized in that the method comprises:
generating a first database with medicine data and associated symptoms of treated patients, wherein the medicine data comprises medicines that are prescribed to the treated patients and an amount of prescribed medicines that are consumed by the treated patients;
generating a second database with medical records of the treated patients, wherein the medical records comprise at least one of medical history, diagnoses by a medical expert, gender, age or genome mapping of the treated patients;
processing an expert input from a medical expert on the medicine data of the treated patients, wherein the expert input comprises feedback associated with the medicine data of the treated patients; and
providing the medicine data, the associated symptoms, the expert input on the medicine data and the medical records of the treated patients to the machine learning algorithm as training data to generate the machine learning model.

16. A method according to claim 15, wherein the method comprises
obtaining a medical record of a new patient, wherein the medical record comprises at least one of medical history, diagnoses by a medical expert, a gender, an age or genome mapping of the new patient;
using the machine learning model to group two or more patients of the same type from the treated patients based on at least one of the gender, the age or the genome mapping of the new patient;
generating a medicine intake matrix by obtaining medicine intake data of two or more medicines in use by the new patient, wherein the medicine intake matrix comprises rows that represent medicines and columns that represent time units when the medicine intake data is measured, and each cell in the medicine intake matrix represents a quantity of medicine used during a time unit, wherein the two or more medicines in use are prescribed by a medical professional for the new patient;
generating a symptom level matrix by obtaining levels of symptoms associated with the two or more medicines from the new patient after intake, wherein the symptom level matrix comprises rows that represent symptoms, columns that represent time units, and each cell in the symptom level matrix represents a level of severity of a symptom;
using the machine learning model to determine a transfer function (F), based on the medicine intake matrix and the symptom level matrix; and
determining, using the transfer function, a prescription comprising an amount of a first medicine and an amount of a second medicine for the new patient to reduce a value of a sum of the symptom level matrix, wherein the transfer function provides an output value of prescription for the new patient based on the medicine intake matrix and the symptom level matrix.

17. A method according to claim 15 wherein the system level matrix is generated using a time series based biosensor data.
